(19) United States
(12) Patent Application Publication
(10) Pub. No.: US 2015/0072436 A1
(43) Pub. Date: Mar. 12, 2015

(54) Methods of Measuring Dissolved Oxygen in a Hydrocarbon Stream

(71) Applicant: Baker Hughes Incorporated, Houston, TX (US)

(72) Inventors: Hua Mo, Friendswood, TX (US); Roger D. Metzler, Sugar Land, TX (US)

(73) Assignee: Baker Hughes Incorporated, Houston, TX (US)

(21) Appl. No.: 14/021,781

(22) Filed: Sep. 9, 2013

(51) Int. Cl. G01N 31/22; G01N 33/28

(52) U.S. Cl. CPC G01N 31/225; G01N 33/2841
USPC 436/138

(57) ABSTRACT

At least one probe may be introduced into a hydrocarbon stream for detecting dissolved oxygen species therein. The hydrocarbon stream may have or include at least one probing additive where the probing additive(s) may be added to the hydrocarbon stream, coated onto the probe(s), or both. The probe(s) may detect at least one luminescent measurement from the hydrocarbon stream, and the luminescent measurement(s) may determine whether dissolved oxygen species are present within the hydrocarbon stream.

### TECHNICAL FIELD

The present invention relates to luminescence techniques for detecting dissolved oxygen in a hydrocarbon stream with at least one probe.

### BACKGROUND

Dissolved oxygen in a hydrocarbon stream causes subsequent reactions within the hydrocarbon stream and potential fouling and/or corrosion of the process equipment and piping, such as peroxide related free radical polymerization etc. ‘Hydrocarbon stream’ is defined herein to include hydrocarbon streams being processed, as well as hydrocarbon streams in tankage or fuels, and the like; such hydrocarbon streams may also be or include hydrocarbon streams in the vapor phase, liquid phase and mixed phase streams. The oxygen intrusion may happen in many ways, such as from a vacuum distillation tower, a cooling water leak, a makeup solvent, storage tank of chemicals, and the like. Dissolved oxygen is a relative measure of the amount of oxygen dissolved or present in a given medium, i.e. oxygen saturation within a hydrocarbon fluid. Oxygen monitoring may be used to trend the oxygen level, or measure the oxygen reactant concentration in order to determine the need for oxygen scavengers, antioxidants, or other oxygen reactive inhibitor additives (hereinafter collectively referred to as ‘oxygen scavengers).

However, measuring the dissolved oxygen in hydrocarbon fluids has been a constant challenge for the petrochemical industry, such as petroleum refining and petrochemical processing. Prior art methods for determining dissolved oxygen concentration in liquid fuel and hydrocarbon process streams are cumbersome and time consuming as to be rendered substantially useless for many measurements. For example, methods based on gas chromatography (GC) and combined GC and mass spectrometry (MS) are sensitive to at least a few parts per million (ppm) O₂ and give results with fairly high precision. However, in the GC based method, the oxygen must be separated from the hydrocarbon prior to introduction to the GC column. Any hydrocarbon in the GC sample also degrades column efficiency, so the column must be run through a heating cycle regularly to remove small amounts of and hydrocarbon process streams. GC does not allow study of rapidly time-varying signals and is performed off-line, which prevents in-situ and spatially resolved sample measurements. Moreover, these methods are expensive to perform.

Electrochemical methods, such as potentiometry and voltammetry, may be used for analysis of oxygen species. Oxygen is detectable with high sensitivity by polarography in aviation fuel because of a paucity of other reducible species in the fuel, but the interface between the fuel and the electrochemical cell is cumbersome and the measurement is slow and cannot be performed non-invasively.

Oxygen is difficult to measure spectroscopically in organic solutions because O₂ does not absorb in the infrared and has electronic transitions in the far ultraviolet spectrum where organic solutions strongly absorb. Although O₂ has a Raman allowed transition and unique electron spin resonance, methods based on these attributes have low sensitivity, high cost and experimental complexity.

Therefore, it would be beneficial to devise quicker and less expensive methods to analyze oxygen species in hydrocarbon fluids with higher rates of repeatability.

### SUMMARY

There is provided, in one form, a method for measuring dissolved oxygen in a hydrocarbon stream. The hydrocarbon stream may be or include, but is not limited to, a mixed C4 hydrocarbon stream, a light ends hydrocarbon stream, a styrene hydrocarbon stream, a refinery fluid, cracked gas distillates, hydrotreator feeds, kerosene, a pyrolysis gas stream, and combinations thereof The hydrocarbon stream may have at least one probe introduced thereinto where the probe(s) are used for detecting oxygen species, and the hydrocarbon stream may have or include at least one probing additive. At least one luminescent measurement may be detected from the hydrocarbon stream with the probe(s) to determine whether at least one oxygen specie is present within the hydrocarbon stream.

In an alternative non-limiting embodiment of the method, the probing additive(s) may be coated onto the probe(s). The probing additive may be or include, but is not limited to platinum, palladium, ruthenium, ytterbium, salts thereof, porphyrins thereof, monoaromatic derivatives thereof, polyaromatic derivatives thereof, and combinations thereof.

In another non-limiting embodiment, the efficacy of an oxygen scavenger present in a hydrocarbon stream may be determined. The hydrocarbon stream may have at least one probe introduced thereinto where the probe(s) are used for detecting oxygen species. The hydrocarbon stream may have or include at least one probing additive and at least one oxygen scavenger. At least one luminescent measurement may be detected from the hydrocarbon stream with the probe(s) to determine the efficacy of the oxygen scavenger(s) present within the hydrocarbon stream.

The probe and probing additives provide a quicker and less expensive mechanism to determine whether oxygen is present in a hydrocarbon stream.

### DETAILED DESCRIPTION

It has been discovered that dissolved oxygen species may be detected within a hydrocarbon stream by introducing at least one probe into the hydrocarbon stream and passing a light therethrough. The hydrocarbon stream may have at least one probing additive present to aid the probe(s) in detecting any dissolved oxygen species. The probe(s) may detect at least one luminescence measurement from the hydrocarbon stream to determine whether oxygen specie(s) are present within the hydrocarbon stream.

Quantifying the amount of oxygen species in the hydrocarbon stream is important for several reasons. When the oxygen species combine with other chemical and/or biological species within the hydrocarbon stream, this may cause fouling and corrosion within the stream and also to the equipment used for handling the hydrocarbon stream. The oxygen measurement may also be used to determine the efficacy of oxygen scavengers for adjusting the dosage of the oxygen scavengers. ‘Oxygen scavenger’ is defined herein to be any compound that targets the oxygen species and reduces or prevents the ability of the oxygen species to react with other species present in the hydrocarbon stream. Oxygen antioxidants, oxygen species inhibitors, or other oxygen reactive inhibitor additives are one non-limiting example of an oxygen scavenger.

By using luminescence measurements to quantify oxygen species in the hydrocarbon stream, rapidly time-varying signals may be measured. The measurements may also be taken on-line or offline, and even at a remote location. The probe may be introduced into a sample extracted from the process, into a slip stream separate from the actual process stream, or directly into the process stream, and the like.

The probing additive(s) may be or include at least one luminophore, such as, but not limited to, platinum, palladium, ruthenium, ytterbium, salts thereof, porphyrins thereof, monoaromatic derivatives thereof, polyaromatic derivatives thereof, and combinations thereof. The salts may be or include, but are not limited to, chlorides, perchlorides, sulfates, bipyridines, and combinations thereof etc. The porphyrins may be or include, but are not limited to, halogenated porphyrins, oxygenated porphyrins, and combinations thereof. The monoaromatic derivative may have a phenyl. The polyaromatics may be or include, but are not limited to naphthalene and anthracene, and combinations thereof.

More generally, luminophores have been used to facilitate optical sensing. As used herein, a “luminophore” is a chemical species that reacts to the presence of a substance to produce an optical result, e.g. a fluorophore. Another type of luminophore changes color in accordance with changes in an amount of a particular substance.

Luminophores may be trapped in a solid substance and deposited as a thin layer or a membrane onto a fiber optic waveguide where the waveguide and the trapped luminophore form a fiber optic probe. The probe may be introduced into the sample to interact with the oxygen species, which results in a change in luminescence properties. This change may be probed and detected through the fiber optic waveguide by an optical detector. The optical detector may be a single photodetector with an optical filter, a spectrometer, or any optical detection system capable of measuring light intensity or the change in light intensity through time. These optical properties of chemical sensor compositions typically involve changes in colors or in color intensities, fluorescence intensity, or fluorescence lifetime.

With these types of probes, it is possible to detect changes in the hydrocarbon streams being monitored at the tip of the fiber sensor by a detector that is located remotely to the sample, in order to thereby provide remote monitoring capabilities. In such systems, the amount of light reaching the detector may limit the sensitivity and signal to noise of the measurement.

In another non-limiting embodiment, fiber optic devices may allow for miniaturization and remote sensing of hydrocarbon streams. The luminophore may be immobilized via mechanical or chemical means to one end of an optical fiber. To the opposite end of the fiber is attached a fiber coupler (Y shaped fiber) or a beam splitter. Incident excitation light may be coupled into one leg of the fiber by a filter and a lens. Excitation light may be carried through the fiber to the distal end where the luminophore is immobilized to the tip.

Upon excitation, the luminophore may uniformly radiate the fluorescent light, some of which is recaptured by the fiber tip and propagated back through the fiber to the junction or “coupler”. At the junction, a substantial portion (typically half) of the fluorescence may be conveyed back to the emitter or point of origin thereby unavailable for signal detection. To offset the inefficiencies of the system, lasers may be used to raise the input power, and highly sensitive photomultiplier tubes may be used as detectors. The other half of the fluorescence may travel along the other leg of the fiber to the detector to be recorded.

The light used in conjunction with the probe to aid in fluorescence of the probing additives may have a wavelength ranging from about 350 nm independently to about 550 nm in another non-limiting embodiment. The probing additive(s) may be added directly to the hydrocarbon stream in a pre-determined amount, coated onto the probe(s), or both. When added to the hydrocarbon stream, the pre-determined amount of the probing additive(s) may range from about 0.1 ppm independently to about 50 vol %, or from about 0.1 ppm independently to about 20 vol %. In another non-limiting embodiment, the probing additive may range from about 0.1 ppm independently to about 10 vol %. In the instance that the probe has the probing additive coated thereonto, and the probing additive has been directly added to the hydrocarbon stream, the amount of probing additive added to the hydrocarbon stream may be much less than the amounts mentioned above. As used herein with respect to a range, “independently” means that any lower threshold may be used together with any upper threshold to give a suitable alternative range.

In one non-limiting embodiment, the probing additive(s) may be coated onto the probe by a sol-gel technique to encapsulate the probing additive sensitive to oxygen species. To coat the probing additive(s) onto the probe with this technique, a fluorinated sol gel precursor [(3,3,3-trifluoropropyl)triethoxysiloxane] may be added to methyltrimethoxysilane (MTMS) for fabricating a multicomponent sol gel medium. Other fluorinated siloxanes precursors may be used, such as but not limited to, (tridecafluoro-1,1,2,2-tetrahydroocyyl)triethoxysilane and (tridecafluoro-1,1,2,2-tetrahydrooctyl)trimethoxysilane.

The multicomponent sol gel may be doped with tris-(4,7-diphenyl-1,10-phenanthroline)ruthenium (II) chloride or other suitable photochemically stable luminophore mentioned above. The doped sol gel may be coated onto optical fibers or other plastic or glass surfaces. To thermally and/or optically cure the coating, the coating may be mixed for three (3) hours, then stored overnight, air aged after coating for one (1) week and then thermal aged at fifty (50) degrees Centigrade overnight.

One non-limiting example of a probe that may be used to detect oxygen in hydrocarbon fluids is the HIOXY probe supplied by Ocean Optics. However, a strong disadvantage to this probe in hydrocarbon fluids is its inability to obtain the same results after probing the same hydrocarbon fluid more than one time. It appears that some of the hydrocarbon fluid and/or the luminophore remain in the sol gel after each use of a probe. The inventors have discovered that vacuuming the sol gel probe after each use pulls any remaining hydrocarbon fluid out of the probe. In addition to the vacuuming or in the alternative, compressed air may be blown onto the probe after each use to remove any remaining hydrocarbon fluid. This added step allows repeated use of the same probe without receiving skewed measurements.

The amount of the oxygen specie(s) within the hydrocarbon stream may range from about 0 ppm independently to about 50 ppm, alternatively from about 0.1 ppm independently to about 20 ppm, or from about 1 ppm independently to about 10 ppm in another non-limiting embodiment. The oxygen species may be dissolved oxygen (O₂) in one non-limiting embodiment.

The hydrocarbon stream may be or include, but is not limited to, a mixed C4 hydrocarbon stream, a light ends hydrocarbon stream, a styrene hydrocarbon stream, a refinery fluid, cracked gas distillates, hydrotreator feeds, kerosene, a pyrolysis gas stream, and combinations thereof and combinations thereof. A C4 hydrocarbon stream may be defined herein to be a butyl-type hydrocarbon chain with 4 carbon atoms. The oxygen in a mixed C4 may form peroxides, initiate free radical polymerization, and/or foul a butadiene process.

A non-limiting example of a ‘mixed C4 hydrocarbon stream’ is a crude butadiene, or a mixed C4 hydrocarbon stream may be one produced from ethylene crackers and contains anywhere from 30 to 80% of 1-3 butadiene, and the like. During the storage and/or transportation of these streams, butadiene may react to form polymers, which can foul flow meters and the transferring pipelines. In addition, polyperoxides may also build up while in storage causing an explosion risk.

‘Light ends’ are defined herein to be or include the lower-boiling components of a mixture of hydrocarbons, such as those evaporated or distilled off easily in comparison to the bulk of the mixture; e.g. ‘light ends’ may be C6 and lighter. Non-limiting examples of ‘light ends’ may be or include, but are not limited to, distillates, such as a straight run distillate, a cracked distillate, and the like.

In one non-limiting embodiment, the hydrocarbon stream may also include an effective amount of at least oxygen scavenger. Non-limiting examples of the oxygen scavengers may be or include, but are not limited to phenylene diamine, phenols, hydroxyl amine, erythobic acid and combinations thereof. The effective amount of the oxygen scavengers within the hydrocarbon stream ranges from about 0.1 ppm independently to about 150 ppm, alternatively from about 1 ppm independently to about 100 ppm, in another non-limiting embodiment.

The efficacy of the oxygen scavenger in the hydrocarbon stream may be tested by measuring the hydrocarbon stream to determine a base-line of oxygen species without the presence of an oxygen scavenger. Then, the oxygen scavenger may be added to the hydrocarbon stream, and the oxygen species may be measured again to compare the oxygen species measurements of the hydrocarbon stream with and without the oxygen scavengers. If the oxygen species measurement from the hydrocarbon stream with the oxygen scavenger(s) is not less than the oxygen species measurement without the scavenger(s), the dosage of the oxygen scavenger may need to be increased or substituted for another type of oxygen scavenger.

The invention will be further described with respect to the following Examples, which are not meant to limit the invention, but rather to further illustrate the various embodiments.

### EXAMPLES

First, the stability of the HIOXY probe was tested in various hydrocarbon streams, such as heptane, hexane, and pentane as model compounds. The test procedures were as follows:
1) The non-calibrated oxygen probe was immersed in the hydrocarbon stream for approximately 20 min and an oxygen species measurement was taken.
2) The probe was taken out of the hydrocarbon stream, and a vacuum was applied to the probe to suck the organic solvent from the probe that was trapped in the porous end of the oxygen tip of the probe.
3) Compressed air was blown onto the probe to blow off any remaining organic solvent.
4) Steps 2 and 3 were repeated three times.
5) The reproducibility of the probe in the air was checked. If about the same measurement was obtained each time, the stability of the probe was acceptable.

After testing the heptane, hexane, and pentane, the factors affecting the results included the lighting of the lab, and the organic solvent used. During the test, the samples were put in a test tube and placed in a dark vessel to avoid any unnecessary light from the lab. Without the vacuum cleaning and air blow step, the results were not reproducible. The organic solvent appeared to be trapped in the porous tip of the probe, so the probe was vacuumed and air blown after each.

With respect to the data in Examples 1-4 below, a reading of 100 vol % in the left-hand column of ‘air’ includes 21 vol % dissolved oxygen. A 100 vol % reading in the right-hand column of ‘hydrocarbon’ may be calculated by using the mole fraction solubility of oxygen in the hydrocarbon (1 atm, 298.15K); i.e. pentane comprises 20.5 vol % oxygen, hexane comprises 20.5 vol % oxygen, heptane comprises 20.55 vol % oxygen. The solubility of oxygen in fluids or liquids is further discussed in the article “The Solubility of Oxygen and Ozone in Liquids”, Battino et al., J. Phys. Chem., Vol. 12, No. 2, 1983; which is herein incorporated by reference in its entirety.

### Example 1

### Heptane

The amount of time for testing the heptane solution and measuring the O₂ saturation was 20 minutes. The average oxygen saturation in the air was 100 cYci with a relative error of <2%. The average reading for oxygen saturation in the heptane was 100% with a relative error of <2%. The small relative error indicated that the probe was stable for short periods of time in the heptane solution.

TABLE 1
Heptane Test Results (% O₂ saturation, calibrated)

| Air | Heptane |
|---|---|
| 99.3 | 97.5 |
| 99.2 | 101.3 |
| 101.2 | 101.1 |

### Example 2

### Hexane

The amount of time for testing the hexane solution and measuring the O₂ saturation was 20 minutes. The average reading in the air was 100% with a relative error of <4%. The average reading for oxygen saturation in the hexane was 100% with a relative error of <2%. The small relative error indicated that the probe was stable at short term in the hexane solution.

TABLE 2
Hexane Test Results (% O₂ Saturation, calibrated)

| Air | Hexane |
|---|---|
| 94.4 | 98.0 |
| 103.9 | 101.5 |
| 101.8 | 100.5 |

### Example 3

### Pentane

The amount of time for testing the pentane solution and measuring the O₂ saturation was 20 minutes. The average reading in the air was 100% with a relative error of <4%. The average reading oxygen saturation in the pentane was 100% with a relative error of <2%. The small relative error indicated that the probe was stable at short term in the pentane solution.

TABLE 3
Pentane Test Results (% O₂ Saturation, calibrated)

| Air | Pentane |
|---|---|
| 97.5 | 97.6 |
| 101.2 | 103.8 |
| 101.3 | 99.4 |

### Example 4

The probe was introduced into a pentane solution that was bubbled with nitrogen. The initial O₂ content of the pentane solution was 100%, but was decreased to 0% oxygen saturated within 1 minute of bubbling the pentane solution with nitrogen.

The probe was introduced into a pentane solution that was bubbled with compressed air. The oxygen content became stable at 100% oxygen saturation in the organic solution within 1 minute. After vacuum cleaning and air blowing the probe after introducing it into the pentane solution, the oxygen content went back to the initial point of 100% oxygen saturation.

In the foregoing specification, the invention has been described with reference to specific embodiments thereof, and has been described as effective in providing methods for measuring oxygen species in a hydrocarbon stream. However, it will be evident that various modifications and changes can be made thereto without departing from the broader spirit or scope of the invention as set forth in the appended claims. Accordingly, the specification is to be regarded in an illustrative rather than a restrictive sense. For example, specific hydrocarbon fluids, luminophores, solvents, salts, and oxygen species falling within the claimed parameters, but not specifically identified or tried in a particular composition or method, are expected to be within the scope of this invention.

The present invention may suitably comprise, consist or consist essentially of the elements disclosed and may be practiced in the absence of an element not disclosed. For instance, the method may consist of or consist essentially of measuring oxygen species in a hydrocarbon stream by passing a light through the hydrocarbon stream, which may be or include, a mixed C4 hydrocarbon stream, a light ends hydrocarbon stream, a styrene hydrocarbon stream, a refinery fluid, cracked gas distillates, hydrotreator feeds, kerosene, a pyrolysis gas stream, and combinations thereof where at least one probe for detecting an amount of oxygen species has been introduced into the hydrocarbon stream; and detecting at least one luminescence measurement from the hydrocarbon stream with the probe(s) to determine whether at least one oxygen specie is present within the hydrocarbon stream.

The words “comprising” and “comprises” as used throughout the claims, are to be interpreted to mean “including but not limited to” and “includes but not limited to”, respectively.

What is claimed is:

1. A method for measuring oxygen species in a hydrocarbon stream comprising:
passing a light through the hydrocarbon stream selected from the group consisting of a mixed C4 hydrocarbon stream, a light ends hydrocarbon stream, a styrene hydrocarbon stream, a refinery fluid, cracked gas distillates, hydrotreator feeds, kerosene, a pyrolysis gas stream, and combinations thereof; wherein at least one probe for detecting an amount of oxygen species has been introduced into the hydrocarbon stream; and wherein the hydrocarbon stream comprises at least one probing additive;
detecting at least a first luminescence measurement from the hydrocarbon stream with the at least one probe to determine whether at least one oxygen species is present within the hydrocarbon stream.

2. The method of claim 1 further comprising adding the at least one probing additive into the hydrocarbon stream in a pre-determined amount ranging from about 0.1 ppm to about 50 vol % prior to the passing a light through the hydrocarbon stream.

3. The method of claim 1, wherein the at least one probing additive is coated onto the at least one probe.

4. The method of claim 1, wherein the at least one probing additive comprises a luminophore selected from the group consisting of platinum, palladium, ruthenium, ytterbium, salts thereof, porphyrins thereof, monoaromatic derivatives thereof, polyaromatic derivatives thereof, and combinations thereof.

5. The method of claim 4, wherein the salts are selected from the group consisting of chlorides, perchlorides, sulfates, bipyridines, and combinations thereof etc., wherein the porphyrins are selected from the group consisting of halogenated porphyrins, oxygenated porphyrins, and combinations thereof; wherein the monoaromatic derivative is phenyl; wherein the polyaromatics are selected from the group consisting of napthalene, anthracene, and combinations thereof; and combinations thereof.

6. The method of claim 1, wherein the hydrocarbon stream further comprises at least one oxygen scavenger.

7. The method of claim 6, wherein the amount of the at least one oxygen scavenger within the hydrocarbon stream ranges from about 0.1 ppm to about 150 ppm.

8. The method of claim 1, wherein the amount of the at least one oxygen species within the hydrocarbon stream ranges from about 0 ppm to about 50 ppm.

9. The method of claim 1, wherein the light has a wavelength ranging from about 350 nm to about 550 nm.

10. The method of claim 1, wherein the at least one oxygen species is dissolved oxygen (O₂).

11. The method of claim 1, further comprising removing excess hydrocarbon fluid from the at least one probe after each luminescence measurement has been detected by a technique selected from the group consisting of vacuuming the at least one probe, contacting the at least one probe with compressed air, and combinations thereof.

12. The method of claim 11, further comprising detecting at least a second luminescence measurement from the hydrocarbon stream with the at least one probe.

13. A method for measuring oxygen species in a hydrocarbon stream comprising:
passing a light through the hydrocarbon stream selected from the group consisting of a mixed C4 hydrocarbon stream, a light ends hydrocarbon stream, a styrene hydrocarbon stream, a refinery fluid, cracked gas distillates, hydrotreator feeds, kerosene, a pyrolysis gas stream, and combinations thereof; wherein at least one probe for detecting an amount of oxygen species has been introduced into the hydrocarbon stream; wherein the at least one probe has a coating of at least one probing additive coated thereon; and wherein the at least one probing additive comprises a luminophore selected from the group consisting of platinum, palladium, ruthenium, ytterbium, salts thereof, porphyrins thereof, monoaromatic derivatives thereof, polyaromatic derivatives thereof, and combinations thereof;
detecting a first luminescence measurement from the hydrocarbon stream with the at least one probe to determine whether at least one oxygen species is present within the hydrocarbon stream;
removing excess hydrocarbon fluid from the at least one probe by a technique selected from the group consisting of vacuuming the at least one probe, contacting the at least one probe with compressed air, and combinations thereof; and
detecting at least a second luminescence measurement from the hydrocarbon stream with the at least one probe.

14. The method of claim 13, wherein the salts are selected from the group consisting of chlorides, perchlorides, sulfates, bipyridines, and combinations thereof etc., wherein the porphyrins are selected from the group consisting of halogenated porphyrins, oxygenated porphyrins, and combinations thereof; wherein the monoaromatic derivative is phenyl; wherein the polyaromatics are selected from the group consisting of napthalene, anthracene, and combinations thereof; and combinations thereof.

15. The method of claim 13, wherein the hydrocarbon stream is selected from the group consisting of a mixed C4 hydrocarbon stream, a light ends hydrocarbon stream, a styrene hydrocarbon stream, a refinery fluid, cracked gas distillates, hydrotreator feeds, kerosene, a pyrolysis gas stream, and combinations thereof.

16. The method of claim 13, wherein the amount of the at least one oxygen specie within the hydrocarbon stream ranges from about 0 ppm to about 50 ppm.

17. A method for determining the efficacy of an oxygen scavenger in a hydrocarbon stream comprising:
passing a light through the hydrocarbon stream selected from the group consisting of a mixed C4 hydrocarbon stream, a light ends hydrocarbon stream, a styrene hydrocarbon stream, a refinery fluid, cracked gas distillates, hydrotreator feeds, kerosene, a pyrolysis gas stream, and combinations thereof; wherein at least one probe for detecting an amount of oxygen species has been introduced into the hydrocarbon stream; wherein the hydrocarbon stream comprises at least one probing additive and at least one oxygen scavenger; and wherein the at least one probing additive comprises a luminophore selected from the group consisting of platinum, palladium, ruthenium, ytterbium, salts thereof, porphyrins thereof, monoaromatic derivatives thereof; polyaromatic derivatives thereof, and combinations thereof; and
detecting a first luminescence measurement from the hydrocarbon stream with the at least one probe; and
comparing the first luminescence measurement to a second luminescent measurement of a hydrocarbon stream that does not have an oxygen scavenger.

18. The method of claim 17, wherein the amount of the at least one oxygen scavenger ranges from about 0.1 ppm to about 150 ppm.
